(19) United States
(12) Patent Application Publication
(10) Pub. No.: US 2021/0068916 A1
(43) Pub. Date: Mar. 11, 2021

(54) METHODS AND SYSTEMS FOR FORCE DETECTION IN ABLATION DEVICES

(71) Applicant: Perceptive Ablation, Inc., Redwood City, CA (US)

(72) Inventors: Bob Sueh-chien Hu, Los Altos Hills, CA (US); Byong-Ho Park, San Jose, CA (US)

(73) Assignee: Perceptive Ablation, Inc., Redwood City, CA (US)

(21) Appl. No.: 16/563,863

(22) Filed: Sep. 7, 2019

(51) Int. Cl. A61B 90/00; A61B 18/00; G01L 5/00

(52) U.S. Cl. CPC A61B 90/06; A61B 18/00; A61B 2018/0212; A61B 2018/00577; G01L 5/0028

(57) ABSTRACT

Apparatuses, methods, and systems for an ablation device (e.g., for catheter-based ablation procedures) that simplifies the invasive treatment of atrial fibrillation by allowing a medical professional to detect both force and pressure during the procedure. For example, using a sensor affixed to the ablation device, a medical professional may detect (or infer) both the force applied to an organ during an ablation procedure as well as the direction of that force. The detected force and direction applied against the surrounding tissue may then be communicated (e.g., using text, on-screen graphics, sounds, etc.) to a medical professional though a display unit connected to the ablation device.

### CROSS-REFERENCE TO RELATED APPLICATIONS

This application claims the benefit of priority of U.S. Provisional Application No. 62/695,080, filed on Jul. 8, 2018. The contents of the foregoing application is incorporated herein in its entirety by reference.

### FIELD OF THE INVENTION

The invention relates to force detection in ablation devices.

### BACKGROUND

Atrial fibrillation is by far the most common arrhythmic heart disorder in the United States. It affects 2 million people and accounts for 500,000 hospital admissions a year. Atrial fibrillation increases the risk of stroke by 5-fold and leads to almost 80,000 deaths annually. While most patients with atrial fibrillation can be managed adequately with conservative medical therapy, a large number of patients develop complications to the medicines used to treat the disease including fatigue, lightheadedness or substantial bleeds. Furthermore, a substantial number of patients are unable to tolerate atrial fibrillation due to disabling symptoms. Drugs that are used to prevent atrial fibrillation are generally not very effective and have potentially serious long-term complications. While open surgical approaches like the Cox-Maze procedure have been shown to be quite effective in treating the disorder, the less invasive catheter-based techniques are typically only successful some 60% of the time.

Catheter ablation has had enormous success in the treatment of many heart rhythm disturbances. In effect, a well-placed destruction of the conductive property of cardiac tissue could interrupt the abnormal conduction pathway which is the basis for essentially all heart arrhythmia. While catheter ablation is highly successful in the treatment of conditions where the offending conduction disturbance is well known and localized such as accessory conduction pathways or atrial flutter, its success in treating the most common arrhythmia of atrial fibrillation has been modest. The procedure frequently requires several hours of anesthesia, radiation, and the use of multiple catheters with their attendant risks even in the hands of skilled operators.

For an ablation procedure to be effect, medical professionals need to apply an appropriate amount of force. Too much force will result in damage to the organ, while too little force will not create enough scar tissue for the ablation procedure to be effective.

### SUMMARY

A device that simplifies the detection and display of the forces and/or directions necessary for the creation of contiguous electrical-isolation lines using a single tip or balloon tip ablation device is described herein. The ablation device improves the success rate of ablation procedures for patients while greatly reducing the costs associated with these procedures. In particular, the apparatuses, methods, and systems described herein relate to an ablation device (e.g., for catheter-based ablation procedures) that simplifies the invasive treatment of atrial fibrillation by allowing a medical professional to detect both a magnitude and direction of an applied force and/or pressure during the procedure. For example, using a sensor affixed to the ablation device, a medical professional may detect (or infer) both the force applied to an organ during an ablation procedure as well as the direction of that force. The detected force and direction applied against the surrounding tissue may then be communicated (e.g., using text, on-screen graphics, sounds, etc.) to a medical professional though a display unit connected to the ablation device.

Because the ablation device features a discrete sensing mechanisms that can detect and provide feedback on the amount of force applied to the distal tip of the ablation device when in contact with tissue, the medical professional may determine whether the distal tip is in direct contact with the tissue as well as the amount of force applied and the direction. The discreteness of the sensing mechanism ensures that the space and freedom inside the organ during intracardiac intervention is not reduced and that target identification and device control is not complicated. Furthermore, as the medical professional now has real-time feedback on the amount of force and direction in which the force is applied, accuracy of the procedure is increased. Additionally, the ablation device uses a sensor mechanism that can be manufactured cheaply, does not require major modifications to other essential elements in modern ablation catheter, and maintains a small profile with respect to the catheter thereby minimizing the real estate necessary to implement the sensor mechanism.

In one aspect, a system for monitoring force during an ablation procedure, may comprise an ablation device for receiving, at a distal end of the ablation device, an applied force. The system may also comprise a sensing mechanism, wherein the sensing mechanism comprises an electrical circuit with a first impedance, and wherein the sensing mechanism is configured to elastically deform in response to receiving the applied force. A first electrical sensor may be included in the sensing mechanism. The first electrical sensor may mechanically switch in response to the elastic deformation. The system may also include control circuitry (e.g., a computer system) configured to determine a second impedance of the electrical circuit in response to the first electrical sensor mechanically switching based on the elastic deformation and determine a direction of the applied force based on the second impedance of the electrical circuit. The control circuitry may then generate for display, on a display device, an indication of the direction.

Various other aspects, features, and advantages of the invention will be apparent through the detailed description of the invention and the drawings attached hereto. It is also to be understood that both the foregoing general description and the following detailed description are examples and not restrictive of the scope of the invention. As used in the specification and in the claims, the singular forms of “a,” “an,” and “the” include plural referents unless the context clearly dictates otherwise. In addition, as used in the specification and the claims, the term “or” means “and/or” unless the context clearly dictates otherwise.

### BRIEF DESCRIPTION OF THE DRAWINGS

FIG. 1 shows an illustrative ablation tip and sensing mechanism, in accordance with one or more embodiments.

FIG. 2 shows an illustrative sensing mechanism featuring a plurality of segments, in accordance with one or more embodiments.

FIG. 3 shows an illustrative plurality of segment struts operating as electro-mechanical switches, in accordance with one or more embodiments.

FIG. 4A shows an illustrative sensing mechanism prior to a force or direction being applied, in accordance with one or more embodiments.

FIG. 4B shows an illustrative sensing mechanism when a first force and a first direction are applied, in accordance with one or more embodiments.

FIG. 4C shows an illustrative sensing mechanism when a second force and a second direction are applied, in accordance with one or more embodiments.

FIG. 5 shows alternative configurations for the structure of the sensing mechanism, in accordance with one or more embodiments.

FIG. 6 shows an illustrative sensing mechanism featuring electrical resistors directly embedded into the sensing mechanism, in accordance with one or more embodiments.

FIG. 7 shows an illustrative ablation device, in accordance with one or more embodiments.

FIG. 8 shows an illustrative graphical user interface for use with the ablation device, in accordance with one or more embodiments.

FIG. 9 shows an illustrative ablation device implementing a balloon electrode mounted on a catheter body, in accordance with one or more embodiments.

FIG. 10 shows an illustrative ablation device implementing a balloon cuff mounted over a rigid electrode on a catheter body, in accordance with one or more embodiments.

FIG. 11 shows a flow chart of illustrative steps for detecting force and direction during an ablation procedure, in accordance with one or more embodiments.

### DETAILED DESCRIPTION OF THE DRAWINGS

In the following description, for the purposes of explanation, numerous specific details are set forth in order to provide a thorough understanding of the embodiments of the invention. It will be appreciated, however, by those having skill in the art that the embodiments of the invention may be practiced without these specific details or with an equivalent arrangement. In other cases, well-known structures and devices are shown in block diagram form in order to avoid unnecessarily obscuring the embodiments of the invention.

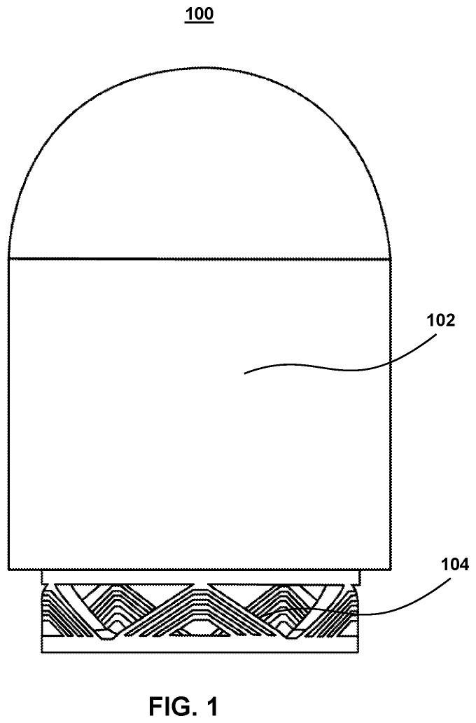

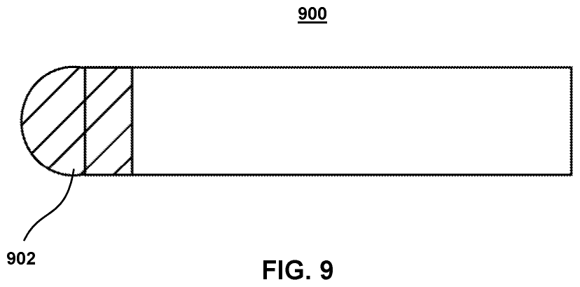

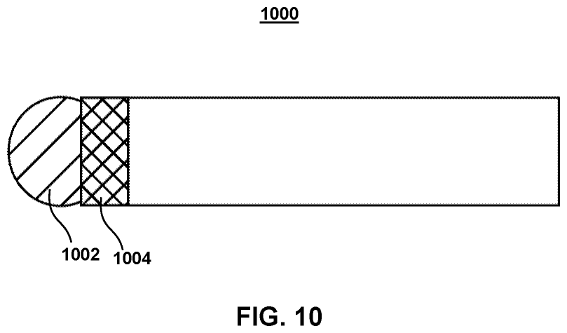

FIG. 1 shows an illustrative ablation tip and sensing mechanism, in accordance with one or more embodiments. Ablation tip 102 is positioned adjacent to sensing mechanism 104 such that a force exerted on ablation tip 102 (e.g., the force exerted by the organ tissue during the ablation procedure in response to the ablation device contacting the tissue) is transferred to the sensing mechanism. In some embodiments, ablation tip 102 may be configured for ablation based on electroporation, radiofrequency, cryotherapy, or laser therapy. Furthermore, in some embodiments, ablation tip 102 may be configured as a balloon ablation tip (e.g., as described in FIGS. 9-10). In some embodiments, ablation tip 102 may use radiofrequency energy to heat tissue around an organ (e.g., a pulmonary vein) in a point-by-point fashion to create a circular scar around areas of the organ (e.g., each vein or around groups of veins). In some embodiments, ablation tip 102 may freeze tissue around the organ to create a scar. In some embodiments, ablation tip 102 may scar tissue around the organ using other energy sources. In some embodiments, ablation tip 102 may use irreversible electroporation to ablate tissue. During electroporation, non-thermal energy can be used to ablate the tissue. In some embodiments, electroporation may be achieved with various modalities: direct current, alternating current, pulsed direct current, or any combination of these.

As shown in FIG. 1, the sensing mechanism 104 is mounted adjacent to ablation tip 102 at the distal end of ablation device 100. In some embodiments, one or more structures and/or materials, which facilitate (and/or do not impede) the transfer of force, may separate ablation tip 102 and sensing mechanism 104. Sensing mechanism 104 is structured in a hollow cylindrical shape and is embedded into ablation device 100 without increasing the overall size of the ablation device. Additionally, as sensing mechanism 104 is hollow, the use of sensing mechanism 104 does not decrease the inner space available for other functionalities such as electrical wiring. For example, sensing mechanism 104 is built into the tubular structure of a catheter, which has a hollow space inside that allows other components to be accommodated for electrical wiring for ablation and mapping, and also irrigation for cooling at the ablation tip.

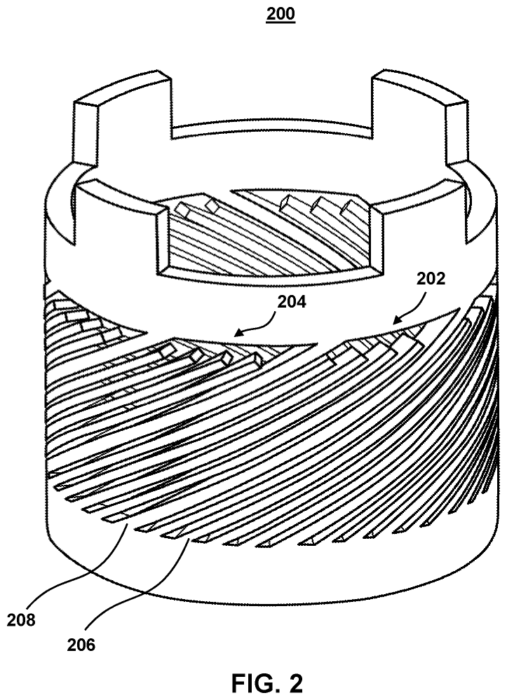

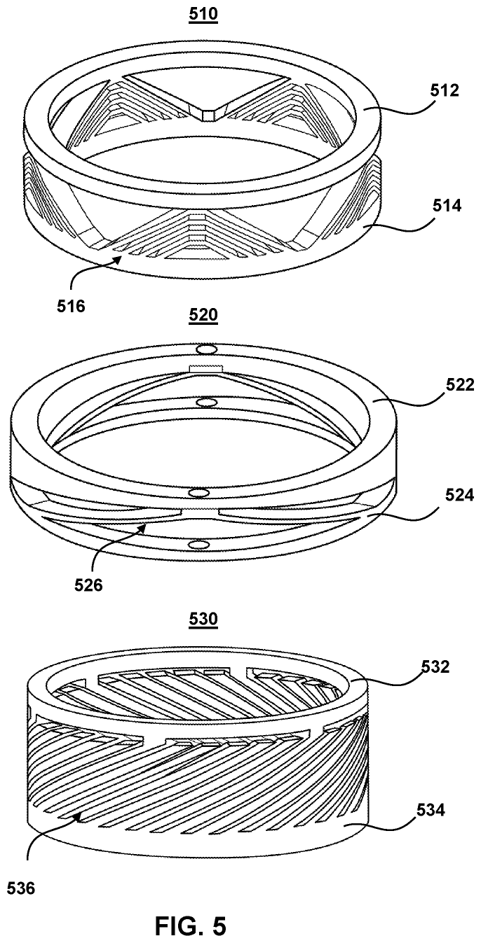

Sensing mechanism 104 includes a pattern of multiple sensors that are mechanically switched. Sensing mechanism 104 may be connected to an external circuit. The powers source of the external circuit may be a battery included within the ablation device or an external power source connected to the ablation device via an electrical line through the catheter. As described below in relation to FIGS. 2 and 5, the pattern of the multiple sensors may include various configurations. In some embodiments, the pattern is represented by a series of repeating segments, which each segment including one or more struts. Each segment may begin with a strut that is bonded and/or in physical contact with a top support structure or a bottom support structure (e.g., support structure 208). In some configurations, each segment may begin with a strut that is bonded and/or in physical contact with at least one of top support structure or bottom support structure multiple times. The pattern corresponding to a segment may repeat multiple times around the circumference of ablation device 100 forming a repeating pattern of segments. It should be noted that in some embodiments, segments on an ablation device may have difference patterns.

Each strut may include one or more sensors that themselves may comprise one or more sets of contacts. The sets of contacts may operate simultaneously, sequentially, or alternately. Each set of contacts may be in an “open” or “closed” state. In an open state, the contacts in the set of contacts are separated, and the switch is not activated (i.e., the switch is nonconducting). In a closed state, the contacts in the set of contacts are touching, and the switch is activated. That is, the switch is conducting, and electricity can flow between the contacts.

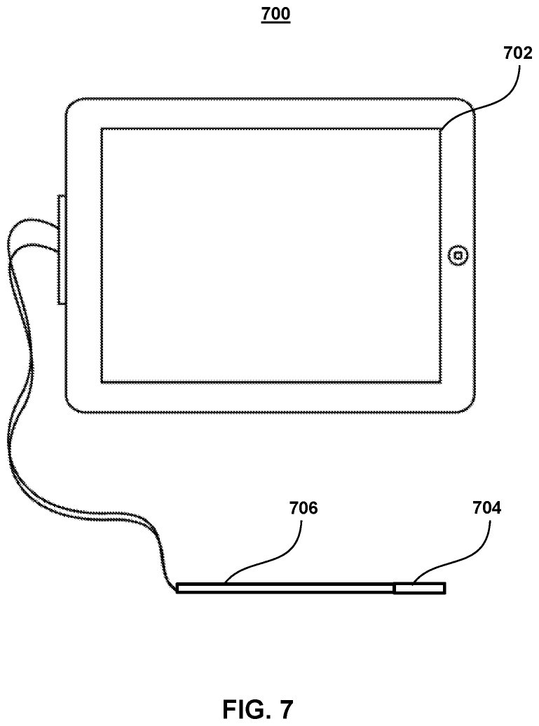

The set of contacts on each strut are mechanically switched in response to mechanical deformation on sensing mechanism 104 that is caused by a force being applied to distal tip 102. For example, in response to distal tip 102 contacting the tissue of the organ, the tissue exerts an equal and opposite force on the distal tip 102. In turn, distal tip 102 exerts a force on sensing mechanism 104. In response to this force, sensing mechanism 104 mechanically deforms through the deformation of one or more segments and/or through the deformation of one or more struts of the one or more segments. This deformation may cause sensors on sensing mechanism 104 to activate as a set of contact that was previously in an open state is now in a closed state due to the deformation. With the set of contacts in the closed state, electricity flows through the sensor as the circuit is complete. The flow of electricity through this sensor is then detected. The sensor may be configured in parallel or in series with other sensors. Through the activation of various sensors in response to the deformation, ablation device 100 (or computer system 702 (FIG. 7)) may detect a deformation (or stress) profile on sensing mechanism 104. Ablation device 100 (or computer system 702 (FIG. 7)) may compare this profile to profile associated with deformations as a result of a direction and magnitude of an applied force. Based on this comparison, ablation device 100 (or computer system 702 (FIG. 7)) may communicate (e.g., to a medical profession performing an ablation procedure) the magnitude and direction of the force applied by the distal tip 104 to the tissue (or the force applied by the tissue to the distal tip 104). Additionally or alternatively, ablation device 100 (or computer system 702 (FIG. 7)) may communicate the profile directly (e.g., in the form of an on-screen graphic) to a medical profession performing an ablation procedure.

FIG. 2 shows an illustrative sensing mechanism featuring a plurality of segments, in accordance with one or more embodiments. For example, in some embodiments, the ablation device may feature a sensing mechanism (e.g., sensing mechanism 200) that features a plurality of segments (e.g., segment 202 and segment 204), each segment may include a plurality of struts (e.g., strut 206). In some embodiments, the length of sensing mechanism 200 may range from 1 mm to 2 mm in some embodiments and may range in length from 0.5 mm to 5 mm depending on the level of resolution detection and sensitivity needed. For example, a length of 1 mm to 5 mm is suitable for detecting a force of 5 to 200 gram-force. In some embodiments, the width of sensing mechanism 200 may range from 50 μm to 100 μm in some embodiments and may range in width from 30 μm to 200 μm depending on the amount of inner space needed within the catheter. Sensing mechanism 200 may comprise one or more materials.

In some embodiments, sensing mechanism 200 may comprise a structure that is molded, extruded, and/or laser cut from one or more materials such as stainless steel, nitinol alloy, and/or another suitable material. For example, sensing mechanism 200 may be the force sensing built out of a single tubular material such as hypodermic tubing that is laser machined to shape.

In some embodiments, the struts in each segment may be substantially the same. For example, the struts may each have substantially the same shape and size, and each segment may feature a plurality of repeating struts. The struts may extend from one end of sensing mechanism 200 to the other at an angle ranging from 10 to 80 degrees with struts at an angle of 20 to 40 degrees for sensing mechanisms requiring a high detection resolution.

Additionally or alternatively, segments in each sensing mechanism may be substantially the same. For example, the segments may each have substantially the same shape, size, and number of struts. For example, sensing mechanism 200 may feature a plurality of repeating segments. The segments (and/or struts) may be arranged circumferentially around the structure of sensing mechanism 202.

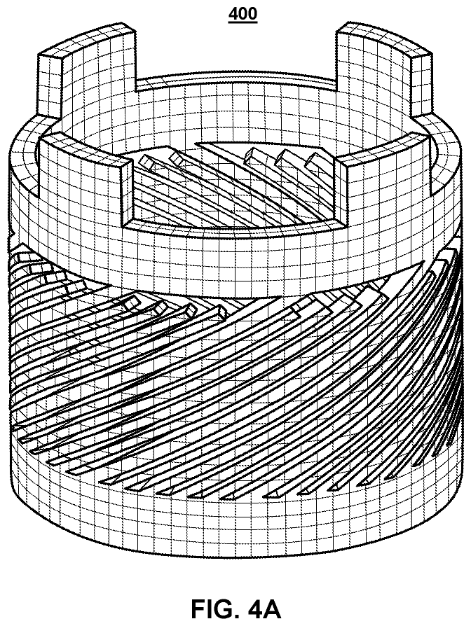

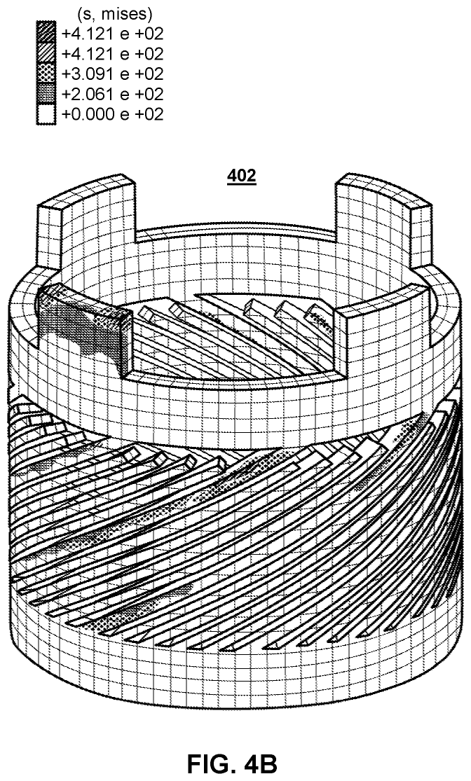

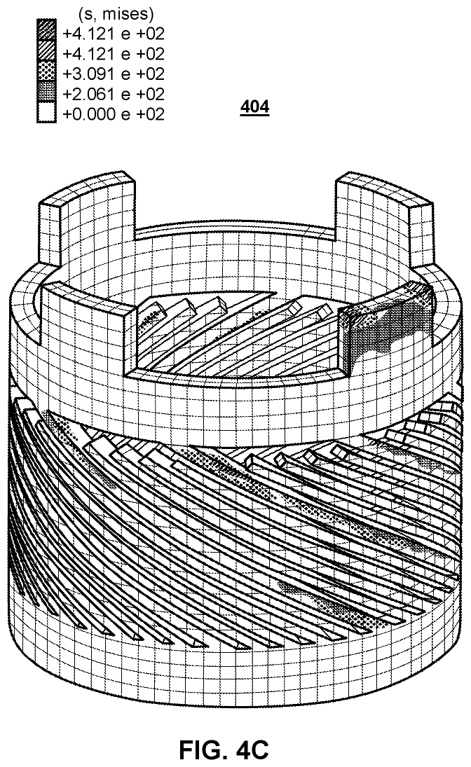

In some embodiments, sensing mechanism 200 may transmit signals based on electrical resistance or mechanical strain to a computer system (e.g., computer system 702 (FIG. 7)), which converts the signals into a format that can be understood by a medical professional. For example, based on the structure of sensing mechanism 200, the force exerted on the tissue may be inferred and the direction of the force may be determined based on the signals as the material properties (e.g., modulus and/or yield strength) of sensing mechanism 200 and/or the dimensions of sensing mechanism 200 create unique profiles (e.g., as described below in FIG. 4A-C) of mechanical strain and/or electrical resistance for different degrees of force and/or directions of applied force.

In some embodiments, a secondary structure such as mechanical stop may be placed within or around sensing mechanism 200 to limit the extent of the deformation and/or deflection. The mechanical stop may be a hollow, cylindrical structure that is shorter in length. The sides of the mechanical stop may include one or more apertures. Alternatively, the mechanical stop may include a first end and a second end. The first end and the second end extending around the inner circumference of the ablation device, and one or more vertical struts may extend from the first end to the second end. In some embodiments, the size and placement of the mechanical stop may be determined based on the size and materials of the sensing mechanism such that the sensing mechanism does not endure excess stress or permanent damage during operation.

It should be noted that the size and shape of the sensing mechanism may be increased and/or decreased in order to accommodate other applications (e.g., in cardiovascular, neurovascular, and/or endovascular medical applications).

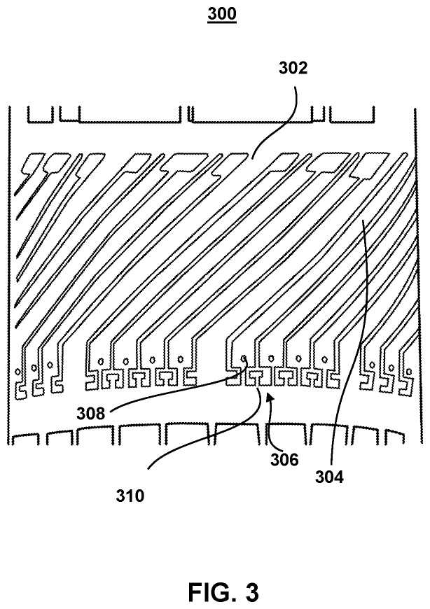

FIG. 3 shows an illustrative plurality of struts operating as electro-mechanical switches, in accordance with one or more embodiments. For example, sensing mechanism 300 may represent an electro-mechanical sensing mechanism. As opposed to sensing mechanisms that rely on detecting mechanical strain, sensing mechanism 300 relies on detecting electrical resistance. Sensing mechanism 300 is composed of support structure 302 and a plurality of struts (e.g., strut 304). Each strut is composed of an array of sensors to detect the amount of force as well as the direction and/or orientation of the force relative to the ablation tip. Once external force is applied to sensing mechanism 300 (e.g., in response to force being exerted on distal tip 104 (FIG. 1)), support structure 302 will start deforming and/or deflecting within an elastic region of the material. Within the strut, each sensor is activated by creating a physical contact with support structure 302 and the other sensors (e.g., on the plurality of struts). It should be noted that as referred to herein a sensor may include a mechanical switch and/or any component that may function as a mechanical switch.

For example, as shown in FIG. 3, strut 304 includes sensor 306. Sensor 306 is currently in an open state (e.g., contact 308 and contact 310 are not touching). Upon strut 304 (or sensing mechanism 300) deforming, contact 308 and contact 310 may activate. In response, sensor 306 transforms to a closed state. An ablation device (e.g., ablation device 100 (FIG. 1) or computer system (e.g., computer system 702 (FIG. 7)) may then determine the impedance on the circuit of sensing mechanism 300. For example, ablation device (e.g., ablation device 100 (FIG. 1) or computer system (e.g., computer system 702 (FIG. 7)) may detect the number of the sensors activated by measuring separate and combined impedance of a circuit comprising sensing mechanism 300.

For example, an ablation device (e.g., ablation device 100 (FIG. 1) or computer system (e.g., computer system 702 (FIG. 7)) may determine the effective resistance of an electric circuit or component to alternating current, arising from the combined effects of ohmic resistance and reactance of each sensor by measuring the separate and combined resistance due to each sensor. For example, electrically each sensor is connected to an electrical resistor (e.g. 10 Ohm, 100 Ohm, 1 k Ohm, 5 k Ohm, 10 k Ohm, 100 k Ohm, 1 M Ohm, 10 M Ohm, 100 M Ohm, etc.) such that each contact creates an electrical short circuit with a preset value of resistance. The more sensors that are activated, the more resistance values are added. Based on the amount of resistance the total force may be determined. Additionally, by determining the location of the sensors that were triggered on the sensing mechanism, the ablation device is also able to determine a direction of the exerted force (e.g., due to the profile of the triggered sensors).

In some embodiments, a secondary structure such as mechanical stop may be placed within or around support structure 302 to limit the extent of the deformation and/or deflection. The mechanical stop may be a hollow, cylindrical structure that is shorter in length. The sides of the mechanical stop may include one or more apertures. Alternatively, the mechanical stop may include a first end and a second end. The first end and the second end extending around the inner circumference of the ablation device, and one or more vertical struts may extend from the first end to the second end. In some embodiments, the size and placement of the mechanical stop may be determined based on the size and materials of the sensing mechanism such that the sensing mechanism does not endure excess stress or permanent damage during operation.

FIG. 4A shows an illustrative sensing mechanism prior to a force or direction being applied, in accordance with one or more embodiments. FIG. 4A shows sensing mechanism 400 at a reference configuration (i.e., a configuration prior to a force being applied to sensing mechanism 400). While at the reference configuration, the plurality of sensors of sensing mechanism 400 (e.g., sensor 306 (FIG. 3)) are in an open state. During an ablation procedure, a force may be applied to sensing mechanism 400 causing sensing mechanism to transition from the reference configuration to a current configuration (as shown in FIGS. 4B and 4C).

FIG. 4B shows an illustrative sensing mechanism when a first force and a first direction are applied, in accordance with one or more embodiments. FIG. 4B illustrates the magnitude of von Mises stress on sensing mechanism 402. For example, sensing mechanism 402 may represent a first current configuration when a 100-gf force is applied at a positive 45 degrees in the longitudinal direction. While at the first current configuration, one or more of the plurality of sensors of sensing mechanism 402 (e.g., sensor 306 (FIG. 3)) have transitioned to the closed state. An ablation device (e.g., ablation device 100 (FIG. 1) or computer system (e.g., computer system 702 (FIG. 7)) may then determine the impedance on the circuit of sensing mechanism 402. For example, ablation device (e.g., ablation device 100 (FIG. 1) or computer system (e.g., computer system 702 (FIG. 7)) may detect the number of the sensors activated by measuring separate and combined impedance of a circuit comprising sensing mechanism 402.

For example, an ablation device (e.g., ablation device 100 (FIG. 1) or computer system (e.g., computer system 702 (FIG. 7)) may determine the effective resistance of an electric circuit or component to alternating current, arising from the combined effects of ohmic resistance and reactance of each sensor by measuring the separate and combined resistance due to each sensor while sensing mechanism 402 is in the first current configuration. Based on the amount of resistance the total force may be determined. Additionally, by determining the location of the sensors that were triggered on sensing mechanism 402, an ablation device (e.g., ablation device 100 (FIG. 1) or computer system (e.g., computer system 702 (FIG. 7)) is also able to determine a direction of the exerted force (e.g., due to the profile of the triggered sensors).

FIG. 4C shows an illustrative sensing mechanism when a second force and a second direction are applied, in accordance with one or more embodiments. FIG. 4C illustrates the magnitude of von Mises stress on sensing mechanism 404. For example, sensing mechanism 404 may represent a second current configuration when a 100-gf force is applied at a negative 45 degrees in the longitudinal direction. While at the second current configuration, one or more of the plurality of sensors of sensing mechanism 404 (e.g., sensor 306 (FIG. 3)) have transitioned to the closed state. An ablation device (e.g., ablation device 100 (FIG. 1) or computer system (e.g., computer system 702 (FIG. 7)) may then determine the impedance on the circuit of sensing mechanism 404. For example, ablation device (e.g., ablation device 100 (FIG. 1) or computer system (e.g., computer system 702 (FIG. 7)) may detect the number of the sensors activated by measuring separate and combined impedance of a circuit comprising sensing mechanism 404.

For example, an ablation device (e.g., ablation device 100 (FIG. 1) or computer system (e.g., computer system 702 (FIG. 7)) may determine the effective resistance of an electric circuit or component to alternating current, arising from the combined effects of ohmic resistance and reactance of each sensor by measuring the separate and combined resistance due to each sensor while sensing mechanism 404 is in the second current configuration. Based on the amount of resistance, the total force may be determined. Additionally, by determining the location of the sensors that were triggered on sensing mechanism 404, an ablation device (e.g., ablation device 100 (FIG. 1) or computer system (e.g., computer system 702 (FIG. 7)) is also able to determine a direction of the exerted force (e.g., due to the profile of the triggered sensors).

FIG. 5 shows alternative configurations for the structure of the sensing mechanism in accordance with one or more embodiments. FIG. 5 includes sensing mechanism 510, 520, and 530. As shown in FIG. 5, each of the embodiments of sensing mechanism 510, 520, and 530 include structures that allow for elastic deformation in both the latitudinal and longitudinal direction. For example, by structuring the sensing mechanism using embodiments that can elastically deform in both the latitudinal and longitudinal direction, the ablation device can detect both the magnitude and the direction of an asserted force. Additionally, while determining the magnitude and the direction of an asserted force based on mechanical compression is less accurate, determining the magnitude and the direction of an asserted force based on electrical resistance is more accurate.

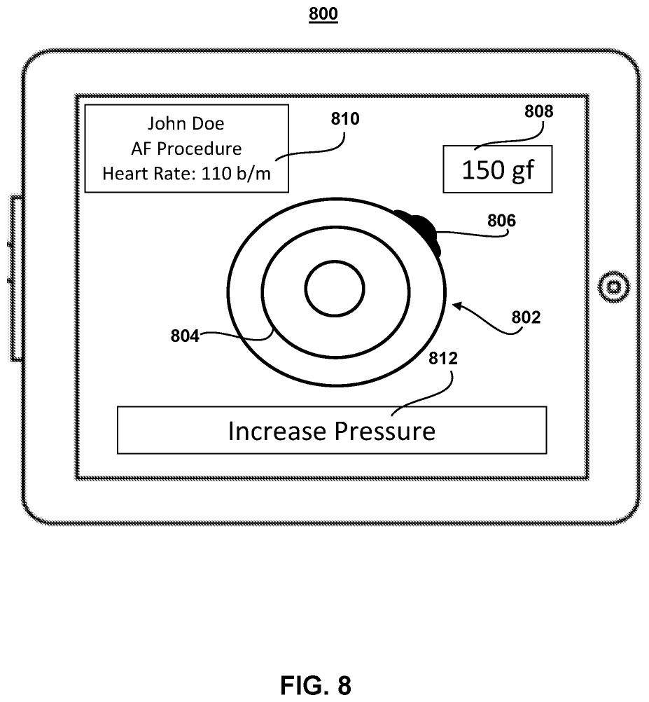

Additional benefits of sensing mechanism using embodiments that can elastically deform in both the latitudinal and longitudinal direction is the increase in potential sensor and sets of contact points. For example, as the sensing mechanism elastically deforms, struts (e.g., strut 304 (FIG. 3)) may touch adjacent struts or support structures. For example, in addition to sensors at the ends of a strut (e.g., as shown in FIG. 3 by contact 308 and 310), points of contact between adjacent struts, support structures, and/or other segments may develop along the length of the strut. These additional points of contact that result from a given deformation (or stress) profile may result in different amounts of electrical resistance being detected. For example, when sensors along the length of a strut are activated, the electrical resistor for that sensor creates an electrical short circuit. That is, the activated sensor creates an abnormal connection in the electrical circuit of the sensing mechanism. The abnormal connection results in different resistances in the electrical circuit of the sensing mechanism. These different resistances may be compared to preset values of resistance (based on known deformation) in order to identify the current deformation (or stress) profile. The current deformation profile may then compare to deformation profiles in a database that lists directions and/or magnitudes associated with a given deformation profile in order to determine the direction and/or magnitude of the current deformation profile. The determined direction and/or magnitude of the current deformation profile may then be communicated to a user via a graphical user interface (e.g., as described in FIG. 8).

The more sensors that are activated, the more resistance values are added. Based on the amount of resistance, the total force may be determined. Additionally, by determining the location of the sensors that were triggered on the sensing mechanism, the ablation device is also able to determine a direction of the exerted force (e.g., due to the profile of the triggered sensors). For example, the activation of a particular pattern of sensors may be correlated to a specific magnitude and/or direction of an exerted force.

Sensing mechanism 510 includes a top support structure 512 and a bottom support structure 514. Sensing mechanism 510 also includes segment 516, which includes four struts. In some embodiments, the number of segments and struts may be based on the resolution and sensitivity required. For example, increasing the number of segments may increase both the resolution and the sensitivity of the device. Additionally, increasing the number of segments (or struts per segment) may increase the maximum amount of force that may be applied prior to failure.

Sensing mechanism 520 includes a top support structure 522 and a bottom support structure 524. Sensing mechanism 520 also includes segment 526, which includes a single strut. While the resolution and sensitivity of sensing mechanism 520 is reduced due to the single strut, manufacturing costs are also decreased. Additionally, top support structure 522 includes a variable width along its length. Sensing mechanism 530 includes a top support structure 532 and a bottom support structure 534. Sensing mechanism 530 also includes segment 536, which includes repeating segments (featuring repeating struts) in a diagonal pattern.

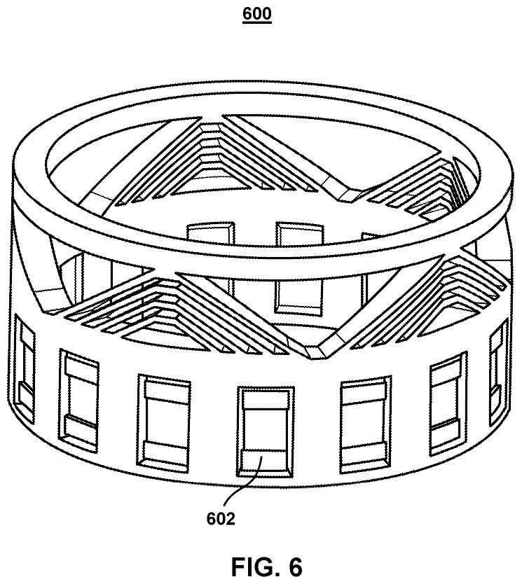

FIG. 6 shows an illustrative sensing mechanism featuring electrical resistors directly embedded into the sensing mechanism, in accordance with one or more embodiments. For example, in some embodiments a thick film chip resistor may be incorporated into the sensing mechanism (e.g., sensing mechanism 600). The thick chip resistors (e.g., resistor 602) may in small sizes (e.g. 01005, 0201, 0402, 0603, etc.) be directly embedded into the force sensing mechanism as shown. The locally embedded or placed resistors reduce the number of electrical wires required to be fed to the ablation device down a catheter line. The electrical resistors can be placed further away within the ablation device or as a part of an external circuitry (e.g., at computer system 702 (FIG. 7)).

FIG. 7 shows system 700 for detecting force and direction during an ablation procedure. As shown in FIG. 1, system 700 may include computer system 702, ablation device 704, catheter 706, and/or other components. It should be noted that the components shown in FIG. 7 are not drawn to scale. Computer system 702 may include a graphical user interface (e.g., as shown in FIG. 8). By way of example, computer system 702 may include a desktop computer, a notebook computer, a tablet computer, a smartphone, a wearable device, or other client device. It should be noted that, while one or more operations are described herein as being performed by particular components of computer system 702, those operations may, in some embodiments, be performed by other components of computer system 702 or other components of system 700.

In some embodiments, the various computers and subsystems illustrated in FIG. 7 may include one or more computing devices that are programmed to perform the functions described herein. The computing devices may include one or more electronic storages, one or more physical processors programmed with one or more computer program instructions, and/or other components. For example, computer system 702 may include control circuitry used to perform one or more computer functions.

The electronic storages may include non-transitory storage media that electronically stores information. The electronic storage media of the electronic storages may include one or both of (i) system storage that is provided integrally (e.g., substantially non-removable) with servers or client devices or (ii) removable storage that is removably connectable to the servers or client devices via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storage may store software algorithms, information determined by the processors, information obtained from servers, information obtained from client devices, or other information that enables the functionality as described herein.

The processors may be programmed to provide information processing capabilities in the computing devices. As such, the processors may include one or more of a digital processor, an analog processor, a digital circuit designed to process information, an analog circuit designed to process information, a state machine, and/or other mechanisms for electronically processing information. In some embodiments, the processors may include a plurality of processing units. These processing units may be physically located within the same device, or the processors may represent processing functionality of a plurality of devices operating in coordination. The processors may be programmed to execute computer program instructions by software; hardware; firmware; some combination of software, hardware, or firmware; and/or other mechanisms for configuring processing capabilities on the processors.

As shown in FIG. 7, ablation device 704 is connected to computer system 702, and computer system 702 includes an external display unit to provide real-time force feedback during use. Catheter 706 extends from ablation device 704 and houses wiring that is fed into computer system 702. It should be noted that catheter 706 may form a rigid or semi-rigid enclosure for one or more wires. In some embodiments, the hollow space inside catheter 706 allows other components to be accommodated such as electrical wiring for powering the ablation, mapping the tissue, and powering the electrical circuit of the sensing mechanism (e.g., sensing mechanism 104 (FIG. 1)). Cather 706 may also accommodate other components for use in the ablation procedure (e.g., irrigation for cooling at the ablation tip). Catheter 706 may be more or less rigid based on the requirements of the ablation procedure. For example, a more rigid catheter 706 may accommodate external pressure sensors (e.g., as discussed in relation to FIGS. 9 and 10 below).

FIG. 8 shows an illustrative graphical user interface for use with the ablation device, in accordance with one or more embodiments. For example, graphical user interface 800 may be incorporated into a computer system (computer system 702 (FIG. 2)) connected to an ablation device (e.g., ablation device 704 (FIG. 7)). It should be noted that the graphic displayed in graphical user interface 800 may include any combination of color, light, sound, and/or alphanumeric characters in order to communicate the magnitude and/or direction of force applied to a sensing mechanism (e.g., sensing mechanism 200 (FIG. 2)).

In FIG. 8, the graphical user interface displays a “bullseye” configuration. In this configuration, graphical indications of the direction of force and magnitude of force appear on graphical user interface 800 as colored portions of ring 802 around a center icon 804, which may in some embodiments represent the ablation target. For example, in response to the direction of force on a first side of the ablation target, corresponding icon 806 appears (e.g., appearing as the colored portion of the ring on the corresponding side). Additionally, numerical indication 808 indicates an amount of force adjacent to corresponding icon 806. These graphic elements allow a medical professional to determine the magnitude and direction of force in a real-time manner.

For example, in response to a force exerted on the distal tip of an ablation device (e.g., distal tip 102 (FIG. 1)), a corresponding force is applied to a sensing mechanism (e.g., sensing mechanism 104 (FIG. 1)) adjacent to the distal tip. The corresponding force causing a mechanical deformation to the sensing mechanism, which mechanically triggers electrical sensors in the sensing mechanism (e.g., as described in FIG. 3). The triggering of these sensors is then displayed in a graphical format on graphical user interface 800.

It should also be noted that in some embodiments, the icons on graphical user interface 800 may be normalized in response to the orientation of the ablation device and/or the ablation target. Graphical user interface 800 may also include user of procedure specific settings (e.g., indications of maximum/minimum force applied to prevent harm to patient, alerts related to maximum/minimum force necessary for procedure, and/or user preferences related to display of information). In some embodiments, graphical user interface 800 may also render images of the tissue and/or overlay information related to the ablation procedure on this image. For example, icon 808 indicates a numerical amount of force being applied. Icon 810 indicates patient specific information and real-time information about the patient. Icon 812 indicates real-time feedback and instructions to the medical professional during the procedure.

FIG. 9 shows an illustrative ablation device implementing a balloon electrode mounted on a catheter body, in accordance with one or more embodiments. FIG. 9 illustrates an embodiment featuring a balloon catheter. For example, a balloon catheter can ablate a larger area of tissue than traditional, single tip ablation devices, which require point-by-point ablation. As shown in FIG. 9, ablation device 900 is configured using balloon ablation tip 902. After inserting the ablation device 900, ablation tip 902 is inflated and the area is ablated using electro, cryo, laser, or radiofrequency energy.

In particular, a balloon comprising polyethylene terephthalate, which provides a high tensile strength and minimal deformability suitable at operations from 1-20 atmospheres of pressure, is used. A balloon comprising polyethylene terephthalate also possess good heat tolerance and transfer with extensive uses in laser and ultrasound applications. In some embodiments, the balloon may be made conductive with silver or other metal coatings.

In some embodiments, when the catheter (e.g., catheter 706 (FIG. 7)) is introduced into a vessel ablation tip 902 may have the same transverse diameter as the catheter. The balloon may then be filled with fluid (e.g., via an irrigation feed within the interior of the catheter. The fluid contained in the ablation tip 902 may be subjected to higher pressure (8-12 atm) causing ablation tip 902 to become harder. Once ablation tip 902 is filled (or have reached a predetermine pressure) an increase in pressure may not lead to a change in size or shape. For example, ablation tip 902 may be constructed to have a preset maximum elasticity in the direction of its diameter. In such cases, the balloon may only have a longitudinal elasticity.

In FIG. 9, ablation tip 902 is constructed using a noncompliant or minimally compliant silver or otherwise conductively coated balloon where the pressure can be monitored simultaneously during an ablation procedure to provide additional safety. For example, ablation tip 902 may feature a noncompliant (e.g., high-pressure) balloon tip comprising polyester or nylon with a conductive coating. Ablation tip 902 may be configured to expand to a specific diameter and exert a specific pressure. Once ablation tip 902 has expanded to the specific diameter and pressure (e.g., upon traversing the patient and arriving at the target organ), the ablation procedure may commence activating the electroporation, radiofrequency, cryotherapy, or laser therapy.

In some embodiments, a sensing mechanism (e.g., sensing mechanism 104 (FIG. 1)) is mounted in series with the pressure sensor such that the sensing mechanism is used to provide directional information on an exerted force, while the pressure sensor is used to quantify the magnitude of the pressure exerted. In some embodiments, the pressure sensor may include a miniature piezoelectric, optical or microelectromechanical element located at the tip or along the body of the ablation device. For example, ablation device 900 may be configured to work with computer system 702 (FIG. 7) and/or any of the sensing mechanism (e.g., as described in FIGS. 1-7 above). Furthermore, graphical user interface 800 may additionally or alternatively indicate the amount of pressure applied. For example, in some embodiments, ablation device 900 may be used to direct the magnitude and/or direction of force and/or pressure. In some embodiments, ablation device 900 may include an external pressure monitor. For example, the pressure monitor may be located outside of ablation device 900 via a catheter. In such embodiments, the catheter (e.g., catheter 706 (FIG. 7)) may be more rigid to ensure pressure sensitivity.

FIG. 10 shows an illustrative ablation device implementing a balloon cuff mounted over a rigid electrode on a catheter body, in accordance with one or more embodiments. Ablation device 1000 features an ablation electrode that is mounted on top of cuff 1004 such that the force experienced by ablation tip 1002 is directly transmitted to the balloon. The balloon pressure is then measured directly for monitoring. Similar to ablation device 900, a pressure sensor of ablation device 1000 may be located outside of ablation device 1000 via a catheter. In such embodiments, the catheter (e.g., catheter 706 (FIG. 7)) may be more rigid to ensure pressure sensitivity. In some embodiments, the pressure sensor may include a miniature piezoelectric, optical or microelectromechanical element located at cuff 1004 or along the body of the ablation device.

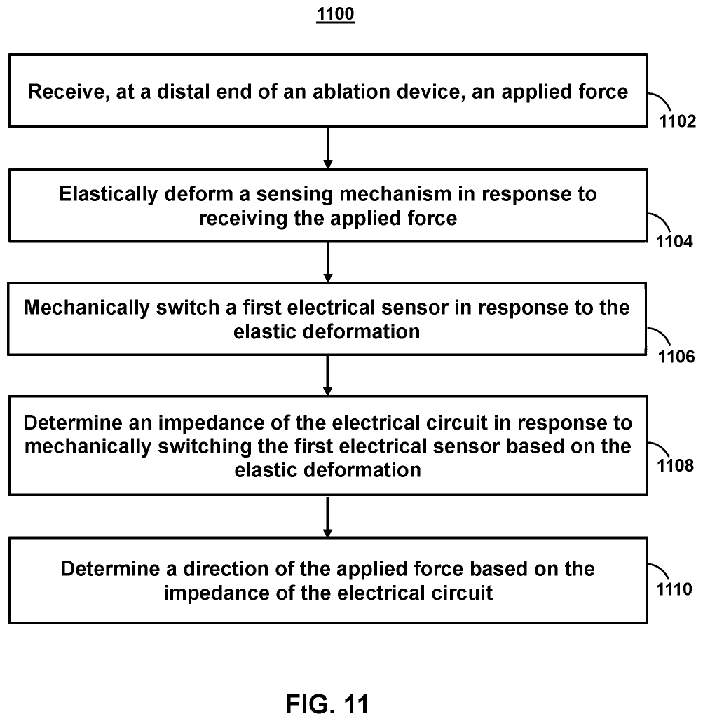

FIG. 11 shows a flow chart of illustrative steps for detecting force and direction during an ablation procedure, in accordance with one or more embodiments. For example, process 1100 may comprise a method for monitoring force during an ablation procedure. It should be noted that one or more steps in process 1100 may be performed by one or more device discussed herein.

At step 1102, an ablation device receives, at a distal end of the ablation device, an applied force. For example, ablation device 100 (FIG. 1) may receive an applied force at its distal end in response to contacting tissue. In some embodiments, the ablation device may include a sensing mechanism (e.g., sensing mechanism 200 (FIG. 2) comprising an electrical circuit with a first impedance. The sensing mechanism may comprise a first support structure that is a first ring and a second support structure that is a second ring. The sensing mechanism may also include one or more segments that extend around the circumference of the first ring to the circumference of the second ring.

In some embodiments, the ablation device may comprise a balloon tip at the distal end (e.g., as described in relation to FIG. 9), and a pressure of the applied force is determined based on a pressure sensor. In some embodiments, the ablation device comprises a balloon cuff at the distal end (e.g., as described in relation to FIG. 10), and a pressure of the applied force is determined based on a pressure sensor in the balloon cuff.

At step 1104, the sensing mechanism may be elastically deformed in response to receiving the applied force. For example, the sensing mechanism may include a first support structure, a second support structure, and a first segment located between the first support structure and the second support structure. The first segment may comprise the first electrical sensor. The first electrical sensor may be mechanically switchable from an open state to a closed state in response to elastic deformation of the first segment. The sensing mechanism may have an electrical circuit with a first impedance when the first electrical sensor is in an open state and has a second impedance when the first electrical sensor is in a closed state.

At step 1106, a first electrical sensor may mechanically switch in response to the elastic deformation. For example, first electrical sensor may comprise a set of contacts that comprise an open state prior to elastic deformation and a closed state after elastic deformation. For example, the first electrical sensor may comprise a set of contacts that do not touch prior to elastic deformation and do touch after elastic deformation. For example, a first contact of the set of contacts may be located on a first strut of a plurality of struts in the first segment, and a second contact of the set of contacts may be located on a second strut of the plurality of struts in the first segment or on the second support structure. During elastic deformation, these contacts may touch.

At step 1108, control circuitry (e.g., computer system 702 (FIG. 7)) may determine a second impedance of the electrical circuit in response to mechanically switching the first electrical sensor based on the elastic deformation. For example, the control circuitry may determine a first separate impedance on the electrical circuit based switching the first electrical sensor. The control circuitry may then determine a second separate impedance on the electrical circuit based switching the second electrical sensor. The control circuitry may then determine a combined impedance on the electrical circuit based switching the first electrical sensor and the second electrical sensor. The control circuitry may then determine the second impedance of the electrical circuit based on the first separate impedance, the second separate impedance, and the combined impedance.

At step 1110, control circuitry (e.g., computer system 702 (FIG. 7)) may determine a direction of the applied force based on the second impedance of the electrical circuit. For example, the control circuitry may determine the direction of the applied force based on the second impedance of the electrical circuit by determining a deformation profile corresponding to the second impedance and determining the direction of the applied force based on the deformation profile.

In some embodiments, the control circuitry may further generate for display, on a display device (e.g., graphical user interface 800 (FIG. 8)), an indication of the direction. In some embodiments, the control circuitry may further determine a magnitude of the applied force based on the second impedance of the electrical circuit and generate for display, on the display device, an indication of the magnitude.

Although the present invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the scope of the appended claims. For example, it is to be understood that the present invention contemplates that, to the extent possible, one or more features of any embodiment can be combined with one or more features of any other embodiment.

The present techniques will be better understood with reference to the following enumerated embodiments:

1. A method comprising: receiving, at a distal end of an ablation device, an applied force, wherein the ablation device includes a sensing mechanism comprising an electrical circuit with a first impedance; in response to receiving the applied force, elastically deforming the sensing mechanism; mechanically switching a first electrical sensor in response to the elastic deformation; in response to mechanically switching the first electrical sensor based on the elastic deformation, determining a second impedance of the electrical circuit; and determining a direction of the applied force based on the second impedance of the electrical circuit.
2. The method of embodiment 1, further comprising determining a magnitude of the applied force based on the second impedance of the electrical circuit.
3. The method of embodiment 2, further comprising mechanically switching a second electrical sensor in response to the elastic deformation, wherein determining the second impedance of the electrical circuit comprises: determining a first separate impedance on the electrical circuit based switching the first electrical sensor; determining a second separate impedance on the electrical circuit based switching the second electrical sensor; determining a combined impedance on the electrical circuit based switching the first electrical sensor and the second electrical sensor; and determining the second impedance of the electrical circuit based on the first separate impedance, the second separate impedance, and the combined impedance.
4. The method of any of embodiments 1-3, wherein determining the direction of the applied force based on the second impedance of the electrical circuit comprises: determining a deformation profile corresponding to the second impedance; and determining the direction of the applied force based on the deformation profile.
5. The method of any of embodiments 1-4, wherein the first electrical sensor comprises a set of contacts that comprise an open state prior to elastic deformation and a closed state after elastic deformation.
6. The method of any of embodiments 1-5, wherein the ablation device comprises a balloon tip at the distal end, and wherein a pressure of the applied force is determined based on a pressure sensor.
7. The method of any of embodiments 1-6, wherein the ablation device comprises a balloon cuff at the distal end, and wherein a pressure of the applied force is determined based on a pressure sensor in the balloon cuff.
8. The method of any of embodiments 1-7, wherein the repair process comprises buffing of one or more portions of the device, pulling on one or more portions of the device, or pushing on one or more portions of the device to mitigate the one or more flaws.
9. The method of any of embodiments 1-8, further comprising generate for display, on a display device, an indication of the direction.
10. The method of any of embodiments 1-9, wherein the sensing mechanism comprises: a first support structure; a second support structure; and a first segment, between the first support structure and the second support structure, comprising a first electrical sensor, wherein the first electrical sensor is mechanically switchable from an open state to a closed state in response to elastic deformation of the first segment, and wherein the sensing mechanism has an electrical circuit with a first impedance when the first electrical sensor is in an open state and has a second impedance when the first electrical sensor is in a closed state.
10. A system comprising: an ablation device, a sensing mechanism, and control circuitry for performing any of embodiments 1-18.

What is claimed is:

1. A system for monitoring force during an ablation procedure, the system comprising:
an ablation device for receiving, at a distal end of the ablation device, an applied force;
a sensing mechanism, wherein the sensing mechanism comprises an electrical circuit with a first impedance, and wherein the sensing mechanism is configured to elastically deform in response to receiving the applied force;
a first electrical sensor in the sensing mechanism that mechanically switches in response to the elastic deformation; and
control circuitry configured to:
determine a second impedance of the electrical circuit in response to the first electrical sensor mechanically switching based on the elastic deformation;
determine a direction of the applied force based on the second impedance of the electrical circuit; and
generate for display, on a display device, an indication of the direction.

2. The system of claim 1, wherein the control circuitry is further configured to:
determine a magnitude of the applied force based on the second impedance of the electrical circuit; and
generate for display, on the display device, an indication of the magnitude.

3. The system of claim 2, further comprising a second electrical sensor, wherein the control circuitry is further configured to determine the second impedance of the electrical circuit by:
determining a first separate impedance on the electrical circuit based switching the first electrical sensor;
determining a second separate impedance on the electrical circuit based switching the second electrical sensor;
determining a combined impedance on the electrical circuit based switching the first electrical sensor and the second electrical sensor; and
determining the second impedance of the electrical circuit based on the first separate impedance, the second separate impedance, and the combined impedance.

4. The system of claim 1, wherein the control circuitry is further configured to determine the direction of the applied force based on the second impedance of the electrical circuit by:
determining a deformation profile corresponding to the second impedance; and
determining the direction of the applied force based on the deformation profile.

5. The system of claim 1, wherein the first electrical sensor comprises a set of contacts that comprise an open state prior to elastic deformation and a closed state after elastic deformation.

6. The system of claim 1, wherein the ablation device comprises a balloon tip at the distal end, and wherein a pressure of the applied force is determined based on a pressure sensor.

7. The system of claim 1, wherein the ablation device comprises a balloon cuff at the distal end, and wherein a pressure of the applied force is determined based on a pressure sensor in the balloon cuff.

8. A method for monitoring force during an ablation procedure, the method comprising:
receiving, at a distal end of an ablation device, an applied force, wherein the ablation device includes a sensing mechanism comprising an electrical circuit with a first impedance;
in response to receiving the applied force, elastically deforming the sensing mechanism;
mechanically switching a first electrical sensor in response to the elastic deformation;
in response to mechanically switching the first electrical sensor based on the elastic deformation, determining a second impedance of the electrical circuit; and
determining a direction of the applied force based on the second impedance of the electrical circuit.

9. The method of claim 8, further comprising determining a magnitude of the applied force based on the second impedance of the electrical circuit.

10. The method of claim 9, further comprising mechanically switching a second electrical sensor in response to the elastic deformation, wherein determining the second impedance of the electrical circuit comprises:
determining a first separate impedance on the electrical circuit based switching the first electrical sensor;
determining a second separate impedance on the electrical circuit based switching the second electrical sensor;
determining a combined impedance on the electrical circuit based switching the first electrical sensor and the second electrical sensor; and
determining the second impedance of the electrical circuit based on the first separate impedance, the second separate impedance, and the combined impedance.

11. The method of claim 8, wherein determining the direction of the applied force based on the second impedance of the electrical circuit comprises:
determining a deformation profile corresponding to the second impedance; and
determining the direction of the applied force based on the deformation profile.

12. The method of claim 8, wherein the first electrical sensor comprises a set of contacts that comprise an open state prior to elastic deformation and a closed state after elastic deformation.

13. The method of claim 8, wherein the ablation device comprises a balloon tip at the distal end, and wherein a pressure of the applied force is determined based on a pressure sensor.

14. The method of claim 8, wherein the ablation device comprises a balloon cuff at the distal end, and wherein a pressure of the applied force is determined based on a pressure sensor in the balloon cuff.

15. An electro-mechanical sensing mechanism for an ablation device, comprising:
a first support structure;
a second support structure; and
a first segment, between the first support structure and the second support structure, comprising a first electrical sensor, wherein the first electrical sensor is mechanically switchable from an open state to a closed state in response to elastic deformation of the first segment, and wherein the sensing mechanism has an electrical circuit with a first impedance when the first electrical sensor is in an open state and has a second impedance when the first electrical sensor is in a closed state.

16. The sensing mechanism of claim 15, wherein the first electrical sensor comprises a set of contacts, and wherein a first contact of the set of contacts is located on a first strut of a plurality of struts in the first segment.

17. The sensing mechanism of claim 15, wherein a second contact of the set of contacts is located on a second strut of the plurality of struts in the first segment.

18. The sensing mechanism of claim 15, wherein a second contact of the set of contacts is located on the second support structure.

19. The sensing mechanism of claim 15, wherein the first segment comprises a plurality of struts, and wherein each of the plurality of struts comprises a respective electrical sensor.

20. The sensing mechanism of claim 15, further comprising a second segment, between the first support structure and the second support structure, comprising a second electrical sensor, wherein the second electrical sensor is mechanically switchable from an open state to a closed state in response to elastic deformation of the second segment, and wherein first segment and the second segment.

21. The sensing mechanism of claim 15, wherein the first support structure is a first ring and the second support structure is a second ring, and wherein the first segment extends from a circumference of the first ring to the circumference of the second ring.
